(19) United States
(12) Patent Application Publication
(10) Pub. No.: US 2025/0375735 A1
(43) Pub. Date: Dec. 11, 2025

(54) SILOXANE TREATMENT SYSTEMS INCLUDING THERMAL HYDROLYZER, CATALYTIC REACTOR AND MEMBRANE SEPARATOR, AND RELATED METHODS

(71) Applicant: GRANITEFUEL ENGINEERING INC., Concord (CA)

(72) Inventors: Shazam S. Williams, Toronto (CA); Linjie Hu, Vaughan (CA)

(21) Appl. No.: 18/874,207

(22) PCT Filed: Aug. 18, 2023
(86) PCT No.: PCT/CA2023/051095

§ 371 Date: Dec. 12, 2024

Related U.S. Application Data
(60) Provisional application No. 63/398,915, filed on Aug. 18, 2022.

(51) Int. Cl. B01D 53/78; B01D 53/22; B01D 53/86

(52) U.S. Cl. CPC B01D 53/78; B01D 53/22; B01D 53/86; B01D 2257/104; B01D 2257/504; B01D 2257/553; B01D 2257/556; B01D 2257/708; B01D 2258/05

(57) ABSTRACT

Systems for and methods of treating a fluid containing siloxanes, silanols, silanes, and/or other silicon compounds. A hot box receives an initial flow of the fluid, and reacts the initial flow with water at a temperature and pressure suitable for hydrolysis to generate a first treated flow, in which at least a portion of the siloxane is hydrolyzed to produce silicon dioxide and methane. A catalytic reactor receives the first treated flow, and converts at least a portion of volatile organic compounds (VOCs) and dioxygen to carbon dioxide and water to generate a second treated flow. A membrane separator receives the second treated flow, and removes at least a portion of the carbon dioxide and water to generate a clean gas flow.

### CROSS REFERENCE TO RELATED APPLICATION

This application claims priority to U.S. Provisional Application No. 63/398,915 filed on Aug. 18, 2022, the entire contents of which are hereby incorporated herein by reference.

### FIELD

The present disclosure relates generally to fluid treatment to remove contaminants.

### BACKGROUND

The following paragraphs are not an admission that anything discussed in them is prior art or part of the knowledge of persons skilled in the art.

Japanese Application No. 2014-83511 A discloses a siloxane decomposition apparatus for removing cyclic siloxane by hydrolysis reaction of cyclic siloxane and water vapor on a catalyst. The siloxane decomposition apparatus introduces a siloxane-containing gas into the siloxane decomposition catalyst from the outside of the apparatus through a gas inlet by being sucked by a suction blower. The gas supplied from the outside is heated by a gas preheater to the temperature required for the hydrolysis reaction to proceed to the temperature of the siloxane decomposition catalyst. Usually, the temperature of the catalyst layer is set in the range of 150 to 300° C., and water vapor is required for the hydrolysis reaction, so that it is supplied from the water supply device to the upstream position of the siloxane decomposition catalyst. Water vapor remaining without being consumed in the reaction is condensed by the cooler and recovered.

U.S. Pat. No. 9,890,674 B2 discloses a method of removing impurities from a gas including the steps of removing impurities from biogas comprising at least one adsorbents via a process vessel or reactor, directing the purified gas to an device to generate power and/or heat, regenerating the saturated adsorption media with the waste heat recovered from the engine exhaust and directing the regeneration gas (hot air or engine exhaust) to flare, engine exhaust stack, or atmosphere.

U.S. application Ser. No. 16/174,814 discloses a filtration apparatus for filtering a fluid stream includes a vessel housing. At least one cartridge assembly is arranged within the vessel housing. The cartridge assembly includes filtration material arranged between at least one inlet and at least one outlet. The filtration material treats the fluid stream to form a filtered fluid stream. In use, the fluid stream is received a feed port of the vessel housing, flows through the filtration material in the cartridge assembly between the inlet and the outlet, and the filtered fluid stream is discharged from a discharge port of the vessel housing. The filtration apparatus can be used to remove siloxanes from the fluid stream.

International Application No. PCT/CA2021/050101 discloses systems for and methods of treating a fluid containing siloxanes, silanes and/or other silicon compounds. A hot box is configured to receive an initial flow of the fluid, react the flow with water at a temperature and pressure suitable for hydrolysis to generate a first treated flow, in which at least a portion is hydrolyzed to produce silicon dioxide and methane, and discharge the first treated flow. A solid removal mechanism can be configured to receive the first treated flow, separate at least a portion of the silicon dioxide as solid material, and discharge the remaining components as a second treated flow.

### INTRODUCTION

The following is intended to introduce the reader to the detailed description that follows and not to define or limit the claimed subject matter.

In an aspect, the present disclosure relates to systems for treating a fluid containing siloxanes, silanols, silanes, and/or other silicon compounds. The system can include: a hot box configured to receive an initial flow of the fluid, and react the initial flow with water at a temperature and pressure suitable for hydrolysis to generate a first treated flow, in which at least a portion of the siloxane is hydrolyzed to produce silicon dioxide and methane; a catalytic reactor configured to receive the first treated flow, and convert at least a portion of volatile organic compounds (VOCs) and dioxygen to carbon dioxide and water to generate a second treated flow; and a membrane separator configured to receive the second treated flow, and remove at least a portion of the carbon dioxide and water to generate a clean gas flow.

The hot box can be operated at a temperature between 250 and 800° C. The hot box can be operated at a temperature between 400 and 450° C.

The catalytic reactor can be operated at a temperature between 200 and 600° C. The catalytic reactor can be operated at a temperature between 300 and 400° C.

The membrane separator can be operated at a temperature between 1 and 95° C. and a pressure between 2 and 50 bar. The membrane separator can be operated at a temperature between 20 and 30° C. and a pressure between 10 and 20 bar.

The system can include at least one heat exchanger configured to transfer heat from the second treated flow to the initial flow. The at least one heat exchanger can increase a temperature of the initial flow to about 250° C.

The system can include a heater configured to heat the initial flow to about 450° C.

The system can include a unit configured to cool, compress, and/or dehydrate the second treated flow.

The fluid can consist of biogas.

In an aspect, the present disclosure relates to methods of treating a fluid containing siloxanes, silanols, silanes, and/or other silicon compounds. The method can include: reacting an initial flow of the fluid with water at a temperature and pressure suitable for hydrolysis to generate a first treated flow, in which at least a portion of the siloxane is hydrolyzed to produce silicon dioxide and methane; with the first treated flow, converting at least a portion of volatile organic compounds (VOCs) and dioxygen to carbon dioxide and water to generate a second treated flow; and with the second treated flow, removing at least a portion of the carbon dioxide and water to generate a clean gas flow.

The step of reacting can include operating a hot box at a temperature between 250 and 800° C. The step of reacting can include operating a hot box at a temperature between 400 and 450° C.

The step of converting can include operating a catalytic reactor at a temperature between 200 and 600° C. The step of converting can include operating a catalytic reactor at a temperature between 300 and 400° C.

The step of removing can include operating a membrane separator at a temperature between 1 and 95° C. and a pressure between 2 and 50 bar. The step of removing can include operating a membrane separator at a temperature between 20 and 30° C. and a pressure between 10 and 20 bar.

The method can include transferring heat from the second treated flow to the initial flow. The method can include increasing a temperature of the initial flow to about 250° C.

The method can include heating the initial flow to about 450° C.

The method can include cooling, compressing, and/or dehydrating the second treated flow.

The fluid can consist of biogas.

Other aspects and features of the teachings disclosed herein will become apparent, to those ordinarily skilled in the art, upon review of the following description of the specific examples of the present disclosure.

### BRIEF DESCRIPTION OF THE DRAWINGS

The drawings included herewith are for illustrating various examples of apparatuses and methods of the present disclosure and are not intended to limit the scope of what is taught in any way. In the drawings:

FIG. 1 is a schematic diagram of a first exemplary system; and

FIG. 2 is a schematic diagram of a second exemplary system.

### DETAILED DESCRIPTION

Various apparatuses or methods will be described below to provide an example of an embodiment of each claimed invention. No embodiment described below limits any claimed invention and any claimed invention may cover apparatuses and methods that differ from those described below. The claimed inventions are not limited to apparatuses and methods having all of the features of any one apparatus or method described below, or to features common to multiple or all of the apparatuses or methods described below. It is possible that an apparatus or method described below is not an embodiment of any claimed invention. Any invention disclosed in an apparatus or method described below that is not claimed in this document may be the subject matter of another protective instrument, for example, a continuing patent application, and the applicant(s), inventor(s) and/or owner(s) do not intend to abandon, disclaim or dedicate to the public any such invention by its disclosure in this document.

The teachings herein relate to an approach for treating fluids, including biogases, that contain siloxanes, silanols, silanes and/or other silicon compounds, and removing such compounds along with oxygen, carbon dioxide and water using an integrated system/method. Firstly, thermal hydrolysis can be used to comprehensively remove the silicon-containing compounds. Secondly, a catalytic reactor can convert volatile organic compounds (VOCs) and dioxygen. Because the catalytic reactor is after thermal hydrolysis, deactivation of catalysis materials downstream by siloxanes can be avoided or at least reduced. Thirdly, a membrane separator can remove carbon dioxide and water. Because the membrane separator is after thermal hydrolysis and the catalytic reactor, membrane damage downstream by siloxanes and/or by VOCs can be avoided or at least reduced.

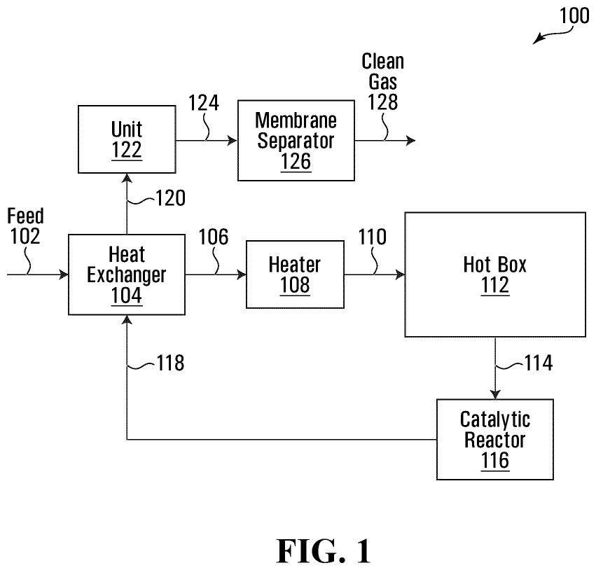

Referring to FIG. 1, a system for treating a fluid containing siloxanes, silanols, silanes and/or other silicon compounds is indicated generally at reference numeral 100. In the system 100, an initial flow 102 of the fluid to be treated is provided. In some examples, the initial flow 102 can consist of raw biogas. In some examples, the fluid can include hydrogen sulfide, organic sulphur molecules, water, oxygen and/or organometallic compounds, including VOCs. In some examples, the initial flow 102 can be delivered at a temperature between 10 and 40° C.

In the example illustrated, the initial flow 102 is received by at least one heat exchanger 104. The heat exchanger 104 can increase its temperature to about 250° C., for example, and generate a first heated flow 106. The first heated flow 106 is then received at a heater 108. The heater 108 can increase its temperature to about 450° C., for example, and generate a second heated flow 110.

In the example illustrated, the second heated flow 110 is received at a thermal hydrolyzer, or “hot box” 112. The hot box 112 can react the flow with water at a temperature and pressure suitable for hydrolysis, in which the siloxane is hydrolyzed and decomposes to produce silicon dioxide and methane, at, for example, between 250 and 800° C., or more specifically between 400 and 450° C., to generate a first treated flow 114 that is discharged from the hot box 112.

In the hot box 112, almost all siloxanes can be hydrolyzed to methane at 400° C. or above, typically >99%. Most silicon compounds can be completely removed, particularly light silicon compounds (silanes, silanol, L2, TMS, etc.). Other siloxane removal technologies can typically only remove heavy siloxanes, such as D4 and D5, and light siloxanes would mostly stay in the feed gas. Silicon-free gas can greatly extend the lifespan of downstream components. Otherwise, light siloxanes can convert to silicon dioxide over precious metal catalyst materials, which can cause fast deactivation of the catalyst and require costly replacement. Furthermore, light siloxanes present in the fluid further downstream can produce deposits on membrane surfaces, causing fouling.

Further details regarding the thermal hydrolyzer/hot box are disclosed in International Application No. PCT/CA2021/050101, the entire contents of which are hereby incorporated herein by reference.

In the example illustrated, the first treated flow 114 is received at a catalytic reactor 116. The catalytic reactor 116 contains oxidation catalyst. The active components in the catalyst can be platinum, palladium, rhodium, ruthenium, or mixtures thereof. The catalyst support can contain aluminum oxide, zirconium oxide, titanium oxide, silicon dioxide, zeolite, activated carbon, or other inorganic materials. The catalyst shape can be pellets, beads, honeycomb, or any other structured substrate. Various configurations are possible.

The catalytic reactor 116 can convert VOCs and dioxygen to carbon dioxide and water, at, for example, between 200 and 600° C., or more specifically between 300 and 400° C., to generate a second treated flow 118 that is discharged from the catalytic reactor 116. The feed gas temperature decreases to about 350° C. due to heat loss. However, because both processes (siloxane and O₂ removal) require approximately the same reaction temperatures (between 300 and 400° C.), energy consumption can be lower compared to two separate processes. Capital expenses can also be reduced for the integrated process, because one set of heater and heat exchanger can be implemented.

The catalytic reactor 116 is downstream of the hot box 112, and therefore the flow 114 received at the catalytic reactor 116 can be substantially free of siloxanes to pass through O₂ removal catalyst materials. This can protect the downstream precious metal catalyst (which can be expensive, e.g., Pt, Pd) for oxygen removal process. O₂ reacts with VOCs to produce CO₂ and water. The O₂ level can be reduced to very low levels, for example, ≤10 ppmv.

In the example illustrated, the second treated flow 118 is received at the heat exchanger 104. In the heat exchanger 104, heat is transferred from the second treated flow 118 to the initial flow 102, to generate a first cooled flow 120.

In the example illustrated, the first cooled flow 120 is received at a unit 122, which can be designed to carry out multiple functions, including cooling, compression, and dehydration, to generate a second cooled flow 124. The unit 122 can perform a number of operation steps, and can consist of several separate units of equipment.

In the example illustrated, the second cooled flow 124 is received at a membrane separator 126. The membrane separator 126 can contain permeation selective membranes, which can selectively separate a H₂O/CO₂-containing gas stream as the permeate and retain a substantially H₂O/CO₂-free gas stream (e.g., a methane-rich gas stream) as the retentate. The separator can include a polymer membrane (e.g., cellulose acetate, polyimide, or combinations thereof), an inorganic membrane (e.g., silica, zeolites, carbon molecular sieves, etc.), and/or a mixed matrix membrane (e.g., a polymeric membrane comprising a dispersed inorganic filler). The membrane separator 126 can be a skid-mounted unit.

The membrane separator 126 removes carbon dioxide and water, at, for example, at a temperature between 1 and 95° C. and a pressure between 2 and 50 bar, or more specifically at a temperature between 20 and 30° C. and a pressure between 10 and 20 bar, to generate a clean gas flow 128 that is discharged from the membrane separator 126. The sources of carbon dioxide and water include those from the raw feed 102, as well as those generated from the catalytic reactor 116 during the oxygen removal process.

The membrane separator 126 is downstream of the hot box 112 and the catalytic reactor 116, and therefore the flow 124 received at the membrane separator 126 can be substantially free of siloxanes and VOCs, which can otherwise cause damage.

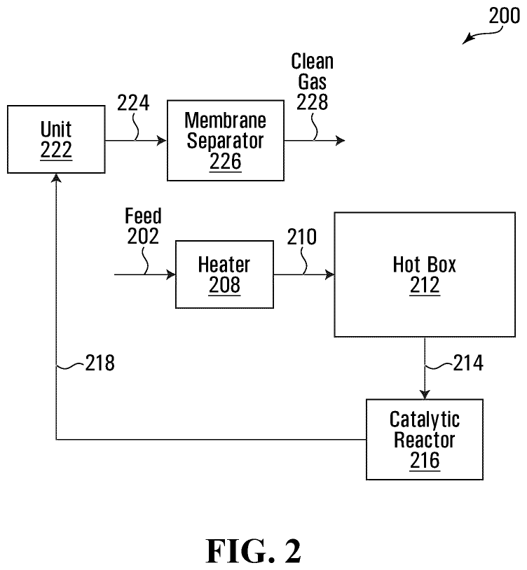

Referring to FIG. 2, a system for treating a fluid containing siloxanes, silanols, silanes and/or other silicon compounds is indicated generally at reference numeral 200. In the system 200, an initial flow 202 of the fluid to be treated is provided. In some examples, the initial flow 202 can consist of raw biogas. In some examples, the initial flow 202 can be delivered at a temperature between 10 and 40° C.

In the example illustrated, the initial flow 202 is received at a heater 208. The heater 208 can increase its temperature to about 450° C., for example, and generate a heated flow 210.

In the example illustrated, the heated flow 210 is received at a hot box 212. The hot box 212 can react the flow with water at a temperature and pressure suitable for hydrolysis, in which the siloxane is hydrolyzed and decomposes to produce silicon dioxide and methane, at, for example, between 400 and 450° C., to generate a first treated flow 214 that is discharged from the hot box 212.

In the example illustrated, the first treated flow 214 is received at a catalytic reactor 216. The catalytic reactor 216 converts volatile organic compounds (VOCs) and dioxygen to carbon dioxide and water, at, for example, between 300 and 400° C., to generate a second treated flow 218 that is discharged from the catalytic reactor 216.

In the example illustrated, the second treated flow 218 is received at a unit 222, which can carry out the functions of cooling, compression, and dehydration, to generate a cooled flow 224.

In the example illustrated, the cooled flow 224 is received at a membrane separator 226. The membrane separator 226 removes carbon dioxide and water, at, for example, at a temperature between 20 and 30° C. and a pressure between 10 and 20 bar, to generate a clean gas flow 228 that is discharged from the membrane separator 226.

The system 100 can be viewed as an integrated process with energy optimization, whereas the system 200 is not energy optimized. The integrated process can save energy, because ˜60% of thermal energy can be recovered after the catalytic reactor 116 with the heat exchanger(s) 104. However, energy recovery can be omitted, as in the system 200, for example, for smaller projects, or if it is beneficial for the clean gas to remain hot for further treatment downstream.

The present disclosure relates generally to the removal of siloxanes and/or other silicon compounds from a fluid stream while reducing or avoiding the emission of waste gases to the environment. Various silicon-containing compounds can be treated, including those in accordance with the exemplary hydrolysis reactions below.

In some examples, cyclic siloxanes can be thermally hydrolyzed according to the following general reaction equation:

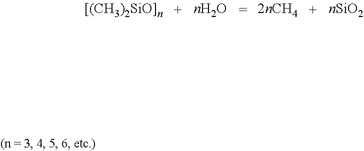

For example, the hydrolysis of octamethylcyclotetrasiloxane (D4):

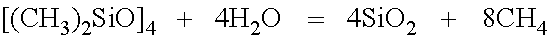

In some examples, linear siloxanes can be thermally hydrolyzed according to the following general reaction equation:

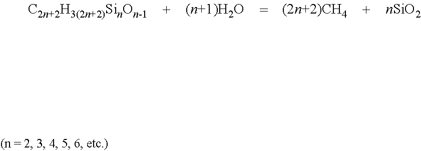

For example, the hydrolysis of hexamethyldisiloxane (L2):

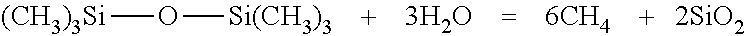

In some examples, silanols can be thermally hydrolyzed, such as trimethylsilanol (TMS) according to the following equation:

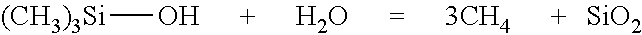

In some examples, silanes can be thermally hydrolyzed, such as methoxytrimethyl silane according to the following equation:

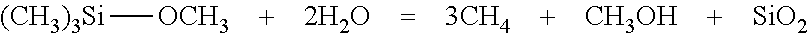

The cleaned gas can be used subsequently in combustion processes, as a renewable natural gas (RNG), to operate fuel cells, etc.

While the above description provides examples of one or more apparatuses or methods, it will be appreciated that other apparatuses or methods may be within the scope of the accompanying claims.

1. A system for treating a fluid containing siloxanes, silanols, silanes, and/or other silicon compounds, comprising:
a hot box configured to receive an initial flow of the fluid, and react the initial flow with water at a temperature and pressure suitable for hydrolysis to generate a first treated flow, in which at least a portion of the siloxanes, silanols, silanes, and/or other silicon compounds is hydrolyzed to produce silicon dioxide and methane;
a catalytic reactor configured to receive the first treated flow, and convert at least a portion of volatile organic compounds (VOCs) and dioxygen to carbon dioxide and water to generate a second treated flow; and
a membrane separator configured to receive the second treated flow, and remove at least a portion of the carbon dioxide and water to generate a clean gas flow.

2. The system of claim 1, wherein the hot box is operated at a temperature between 250 and 800° C.

3. The system of claim 1, wherein the hot box is operated at a temperature between 400 and 450° C.

4. The system of claim 1, wherein the catalytic reactor is operated at a temperature between 200 and 600° C.

5. The system of claim 1, wherein the catalytic reactor is operated at a temperature between 300 and 400° C.

6. The system of claim 1, wherein the membrane separator is operated at a temperature between 1 and 95° C. and a pressure between 2 and 50 bar.

7. The system of claim 1, wherein the membrane separator is operated at a temperature between 20 and 30° C. and a pressure between 10 and 20 bar.

8. The system of claim 1, comprising at least one heat exchanger configured to transfer heat from the second treated flow to the initial flow.

9. The system of claim 8, wherein the at least one heat exchanger increases a temperature of the initial flow to about 250° C.

10. The system of claim 1, comprising a heater configured to heat the initial flow to about 450° C.

11. The system of claim 1, comprising a unit configured to cool, compress, and/or dehydrate the second treated flow.

12. The system of claim 1, wherein the fluid consists of biogas.

13. A method of treating a fluid containing siloxanes, silanols, silanes, and/or other silicon compounds, comprising:
reacting an initial flow of the fluid with water at a temperature and pressure suitable for hydrolysis to generate a first treated flow, in which at least a portion of the siloxanes. silanols, silanes, and/or other silicon compounds is hydrolyzed to produce silicon dioxide and methane;
with the first treated flow, converting at least a portion of volatile organic compounds (VOCs) and dioxygen to carbon dioxide and water to generate a second treated flow; and
with the second treated flow, removing at least a portion of the carbon dioxide and water to generate a clean gas flow.

14. The method of claim 13, comprising, in the step of reacting, operating a hot box at a temperature between 250 and 800° C.

15. The method of claim 13, comprising, in the step of reacting, operating a hot box at a temperature between 400 and 450° C.

16. The method of claim 13, comprising, in the step of converting, operating a catalytic reactor at a temperature between 200 and 600° C.

17. The method of claim 13, comprising, in the step of converting, operating a catalytic reactor at a temperature between 300 and 400° C.

18. The method of claim 13, comprising, in the step of removing, operating a membrane separator at a temperature between 1 and 95° C. and a pressure between 2 and 50 bar.

19. The method of claim 13, comprising, in the step of removing, operating a membrane separator at a temperature between 20and 30° C. and a pressure between 10 and 20 bar.

20. The method of claim 13, comprising transferring heat from the second treated flow to the initial flow.

21. The method of claim 20, comprising increasing a temperature of the initial flow to about 250° C.

22. The method of claim 13, comprising heating the initial flow to about 450° C.

23. The method of claim 13, comprising cooling, compressing, and/or dehydrating the second treated flow.

24. The method of claim 13, wherein the fluid consists of biogas.

25. (canceled)
